(19) United States
(12) Patent Application Publication
(10) Pub. No.: US 2009/0162252 A1
(43) Pub. Date: Jun. 25, 2009

(54) Incense powder base assembly

(76) Inventors: Chun-Ti Chang, Taichung Hsien (TW); Yung-Tai Chang, Taichung Hsien (TW)

Correspondence Address: CHARLES E. BAXLEY, ESQUIRE, 90 JOHN STREET, SUITE 309, NEW YORK, NY, 10038, US

(21) Appl. No.: 12/316,140

(22) Filed: Dec. 10, 2008

(30) Foreign Application Priority Data
Dec. 24, 2007 (TW) 096221980

(51) Int. Cl. A61L 9/03

(52) U.S. Cl. 422/123

(57) ABSTRACT

An incense powder base assembly has a base, a spacer and a powder mold. The spacer is detachably attached to the top of the base and is annular to form a central hole. The powder mold is detachably attached to the spacer and has a powder channel tortuously defined through the powder mold and having multiple turning points. Accordingly, a separating layer is formed on the top of the base to keep oil sludge generated during the burning of incense from permeating into the base, so the base assembly is easily cleaned and the appearance of the base assembly is neat.

### BACKGROUND OF THE INVENTION

1. Field of the Invention

The present invention relates to an incense powder base assembly, and more particularly to an incense powder base assembly that is easily assembled and cleaned.

2. Description of Related Art

TW M307362 Utility Model, entitled to “Incense Powder Base Assembly” disclosed a conventional incense powder base assembly comprising a base and a mold. The mold is directly attached to the top of the base, and each base and mold has a powder groove. With such an arrangement, a heat-insulated layer and an incense layer are formed based on the grooves. The incense layer is easily ignited and completely burned, and the base assembly is convenient in use.

However, when the incense layer is burned, oil sludge will generate and permeate into the heat-insulated layer held in the groove in the base. Consequently, to clean the heat-insulated layer is difficult and troublesome to cause the appearance of the base assembly unclean.

To overcome the shortcomings, the present invention tends to provide an incense powder base assembly to mitigate or obviate the aforementioned problems.

### SUMMARY OF THE INVENTION

The main objective of the invention is to provide an incense powder base assembly that is easily cleaned. The incense powder base assembly comprises a base, a spacer and a powder mold. The spacer is detachably attached to the top of the base and is annular to form a central hole. The powder mold is detachably attached to the spacer and comprises a powder channel tortuously defined through the powder mold and having multiple turning points.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of an incense powder base assembly in accordance with the present invention;

FIG. 2 is a partially exploded perspective view of the incense powder base assembly in FIG. 1;

FIG. 3 is an operational side view in partial section of the incense powder base assembly in FIG. 1;

FIG. 4 is an operational side view in partial section of the incense powder base assembly in FIG. 3 showing that the spacer and mold are detached from the base;

FIG. 5 is an operational side view in partial section of the incense powder base assembly in FIG. 1 showing that the base assembly with the cap is in use; and

FIG. 6 is an operational side view in partial section of the incense powder base assembly in FIG. 1 showing that ashes on the base are cleaned.

### DETAILED DESCRIPTION OF PREFERRED EMBODIMENT

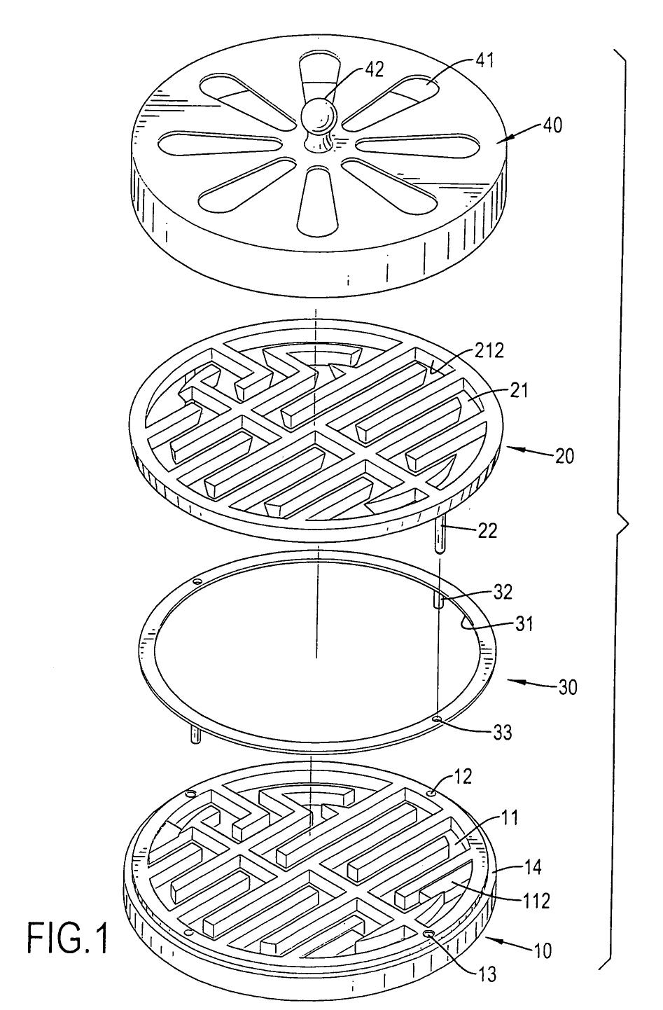

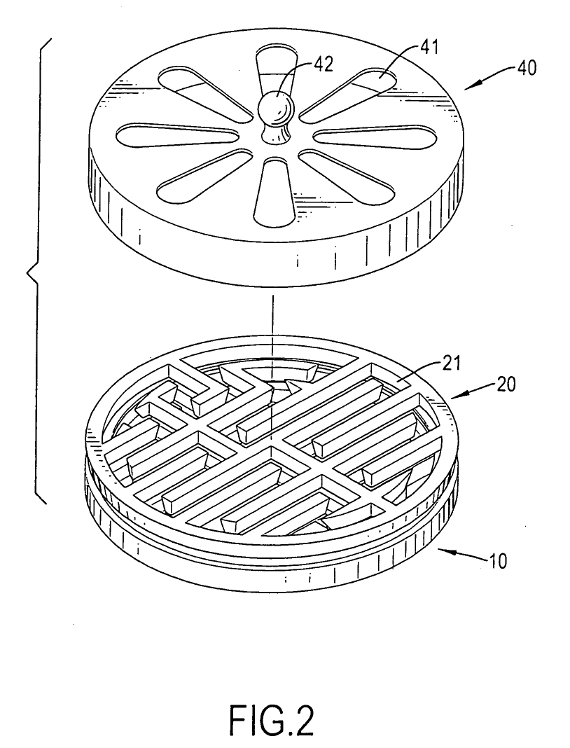

With reference to FIGS. 1 to 3, an incense powder base assembly in accordance with the present invention comprises a base (10), a spacer (30), a powder mold (20), and a cap (40).

The base (10) may be a square, rectangular or round disk and has a top, a bottom, a powder groove (11), multiple rod holes (12) and a shoulder (14). The powder groove (11) is tortuously defined in the top and has multiple turning points. The groove (11) may further have multiple protrusions (112) each formed on one of the turning points to reduce the depth of the groove (11) at the turning point. The rod holes (12) are defined in the top of the base (10) around the powder groove (11). In a preferred embodiment, the rod holes (12) are defined through the base (10) from the top to the bottom. The shoulder (14) is formed around the top of the base (10).

The spacer (30) is attached detachably to the top of the base (10), is annular to define a central hole (31) and has a top, a bottom, multiple connecting rods (32) and multiple rod holes (33). The central hole (31) encloses the tortuous powder groove (11) in the base (10). The connecting rods (32) are formed and protrude from the bottom of the spacer (30) and are inserted respectively into some of the rod holes (12) in the base (10) to make the spacer (30) securely held at the top of the base (10). The rod holes (33) are defined through the spacer (30) and align with the rod holes (12) in the base (10) that are kept free from the connecting rods (32).

The powder mold (20) is attached to the top of the spacer (30) and comprises a top, a bottom, a powder channel (21) and multiple connecting rods (22). The powder channel (21) is tortuously defined through the mold (20) and has a shape corresponding to that of the powder groove (11) in the base (10), multiple turning points and an inner surface (212). The inner surface (212) of the powder channel (21) may be inclined at an angle of 5° and has a width gradually decreasing from the bottom to the top of the mold (20). The connecting rods (22) are formed on and protrude from the bottom of the mold (20) and are inserted respectively into the aligning and corresponding rod holes (12,33) in the spacer (30) and base (10) to securely attach the mold (20) at the top of the spacer (30). Each connecting rod (32,22) on the spacer (30) and the mold (20) has a diameter smaller than that of the corresponding rod hole (12,33) in the base (10) and spacer (30).

The cap (40) is attached to the base (10) to enclose the spacer (30) and the powder mold (20) inside and has a top, a knob (42) and multiple vents (41). The knob (42) is mounted on the top of the cap (40) to allow a user to take the cap (40) with the knob (42). The vents (41) are defined through the cap (40) and correspond to the powder channel (21) and groove (11).

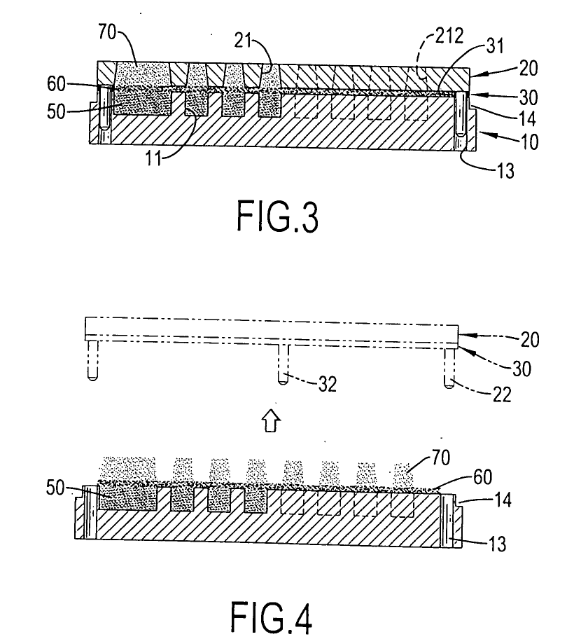

In use, with reference to FIGS. 3 and 4, the powder groove (11) in the base is firstly filled with ashes or powders to form a heat-insulated layer (50) inside the groove (11). The spacer (30) is then attached to the top of the base (10), and the central hole (31) of the spacer (30) is filled with incense powder or ashes to form a separating layer (60) inside the central hole (31). The powder mold (2)) is attached to the top of the spacer (30), and the powder channel (21) is filled with incense powder to form an incense layer (70) in the mold (20) and on the separating layer (60) based on the shape of the channel (21). After the incense layer (70) is formed, the mold (20) and spacer (30) are detached from the base (10). With the inclined inner surface (212) of the powder channel (21), the incense layer (70) has a cross section with a wider bottom but a thinner top and the mold (20) is easily detached from the base (10). With the shape of the cross section of the incense layer (70), the burning speed of the incense layer (70) is even and uniform.

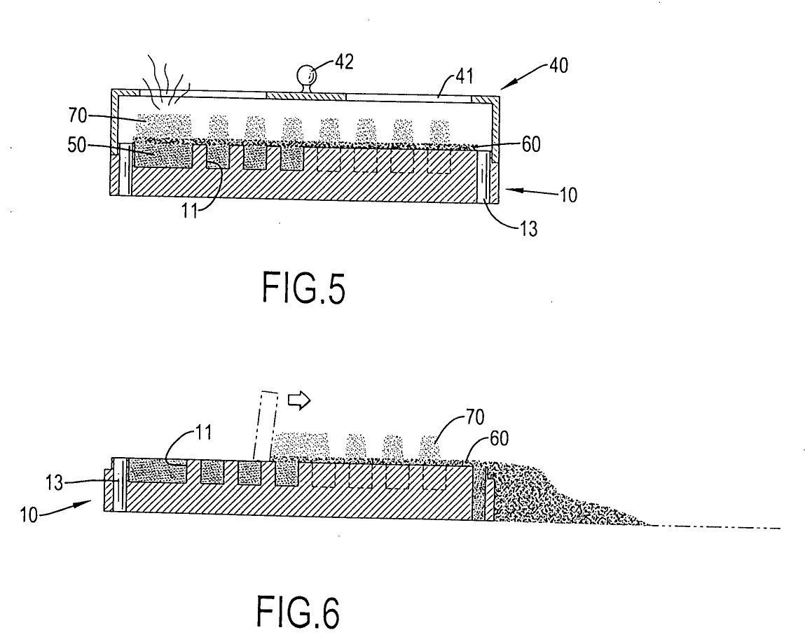

With further reference to FIGS. 5 and 6, the incense layer (70) is ignited to generate fragrant smoke, and the cap (40) is attached to base (10) and supported by the shoulder (14) to allow the fragrant smoke to spread out from the vents (41) in the cap (40).

Because a separating layer (60) is formed between the heat-insulated layer (50) and the incense layer (70), oil sludge generated during the burning of the incense layer (70) is kept from permeating into the heat-insulated layer (50) held inside the powder groove (11) in the base (10). Accordingly, ashes generated by the burned incense powder is located on the top of the base (10) and can be easily cleaned with a scraper or any appropriate tool, and the heat-insulated powder layer (50) is also easily cleaned even can be used repeatedly.

What is claimed is:

1. An incense powder base assembly comprising:
a base having a top and a bottom;
a spacer detachably attached to the top of the base, being annular to form a central hole and having a top and a bottom; and
a powder mold detachably attached to the top of the spacer and comprising a powder channel tortuously defined through the powder mold and having multiple turning points.

2. The incense powder base assembly as claimed in claim 1 further comprising a cap attached to the base to enclose the spacer and powder mold inside.

3. The incense powder base assembly as claimed in claim 2, wherein the powder channel has an inclined inner surface having a width gradually decreasing from a bottom to a top of the powder mold.

4. The incense powder base assembly as claimed in claim 3, wherein the inner surface of the powder channel is inclined at an angle of 5°.

5. The incense powder base assembly as claimed in claim 4, wherein the cap further has
a knob mounted on a top of the cap; and
multiple vents defined through the cap and correspond to the powder channel in the powder mold.

6. The incense powder base assembly as claimed in claim 5, wherein the base further has
a powder groove tortuously defined in the top of the base and having a shape corresponding to that of the powder groove in the powder mold and multiple turning points; and
a shoulder formed around the top of the base to support the cap.

7. The incense powder base assembly as claimed in claim 6, wherein
the base further has multiple rod holes defined in the top of the base;
the spacer further has
multiple connecting rods formed on and protruding from the bottom of the spacer and inserted respectively into some of the rod holes in the base; and
multiple rod holes defined through the spacer and aligning with the rod holes in the base that are kept free from the connecting rods on the spacer; and
the powder mold further has multiple connecting rods formed on and protruding from a bottom of the powder mold and inserted respectively into the aligning rod holes in the spacer and base.

8. The incense powder base assembly as claimed in claim 7, wherein the powder groove in the base further has multiple protrusions each formed on one of the turning points of the powder groove.

9. The incense powder base assembly as claimed in claim 8, wherein the rod holes in the base are defined through the base from the top to the bottom.

10. The incense powder base assembly as claimed in claim 1, wherein the powder channel has an inclined inner surface having a width gradually decreasing from a bottom to a top of the powder mold.

11. The incense powder base assembly as claimed in claim 10, wherein the inner surface of the powder channel is inclined at an angle of 5°.

12. The incense powder base assembly as claimed in claim 2, wherein the cap further has
a knob mounted on a top of the cap; and
multiple vents defined through the cap and correspond to the powder channel in the powder mold.

13. The incense powder base assembly as claimed in claim 12, wherein the base further has
a powder groove tortuously defined in the top of the base and having a shape corresponding to that of the powder groove in the powder mold and multiple turning points; and
a shoulder formed around the top of the base to support the cap.

14. The incense powder base assembly as claimed in claim 1, wherein
the base further has multiple rod holes defined in the top of the base;
the spacer further has
multiple connecting rods formed on and protruding from the bottom of the spacer and inserted respectively into some of the rod holes in the base; and
multiple rod holes defined through the spacer and aligning with the rod holes in the base that are kept free from the connecting rods on the spacer; and
the powder mold further has multiple connecting rods formed on and protruding from a bottom of the powder mold and inserted respectively into the aligning rod holes in the spacer and base.

15. The incense powder base assembly as claimed in claim 14, wherein the rod holes in the base are defined through the base from the top to the bottom.

16. The incense powder base assembly as claimed in claim 1, wherein the powder groove in the base further has multiple protrusions each formed on one of the turning points of the powder groove.
